(19) United States
(12) Patent Application Publication
(10) Pub. No.: US 2020/0289418 A1
(43) Pub. Date: Sep. 17, 2020

(54) METHOD FOR INCREASING BIOAVAILABILITY OF NATURAL BIOACTIVE MOLECULES USING BIO-POLYMER COMPLEXATION TECHNOLOGY

(71) Applicant: ADSO NATURALS PRIVATE LIMITED, Bangalore (IN)

(72) Inventor: Sreerag Gopi, Bangalore (IN)

(21) Appl. No.: 16/817,438

(22) Filed: Mar. 12, 2020

(30) Foreign Application Priority Data
Mar. 12, 2019 (IN) 201941009642

(51) Int. Cl. A61K 9/14; A61K 9/48; A61K 9/00; A61K 36/88; A61K 36/185; A61K 36/28; A61K 33/04

(52) U.S. Cl. CPC A61K 9/146; A61K 9/4866; A61K 9/0053; A61K 33/04; A61K 36/185; A61K 36/28; A61K 36/88

(57) ABSTRACT

A method for increasing bioavailability of natural bioactive molecule using biopolymer complexation technology is provided. The method includes integrating the natural bioactive molecule in the form of nutraceuticals formulations, antioxidants, supplements, and herbs with the cellulose derivatives by modifying surface of the cellulose derivatives with the natural bioactive molecule through hydrogen bonding and ionic interaction between functional groups and forming a complex of natural bioactive molecule and cellulose derivatives. The complex of natural bioactive molecule and cellulose derivatives absorbed in to blood stream as a single entity and dissociates once it is absorbed that subsequently enhances the bioavailability of the natural bioactive molecule.

### CROSS-REFERENCE TO PRIOR FILED PATENT APPLICATIONS

This patent application claims priority to the Indian provisional application no. 201941009642 filed on Mar. 12, 2019 the complete disclosure of which, in its entirely, is herein incorporated by reference.

### BACKGROUND

### Technical Field

The embodiment herein generally relates to a biopolymer complexation technology, and more particularly to a method for increasing the bioavailability of the natural bioactive molecules by complexing it with cellulose derivatives.

### Description of the Related Art

Most of the natural products obtained from plants contain high bioactive molecules which can be considered for therapeutic actions. The effectiveness of the bioactive molecules is mainly dependent upon the form in which it is delivered. Most of the plant constituents are water soluble and that is one of the main reasons for their poor bioavailability, inability to cross the lipid membranes of the intestine. When the natural product comprising, the bioactive molecules are used, it is essential to reach the bioactive molecules in systemic circulation, otherwise, the bio-efficiency of the bioactive molecules may be limited. There are several attempts have been made to deliver bioactive molecules across the barriers.

It is known in the art, the bioavailability of the natural bioactive molecules may be improved by complexing it with inert substances like phospholipids. The use of phospholipids with natural bioactive molecules enhances a rate of release as well as a capacity to cross the lipid rich bio-membranes. Complexation with phospholipids-based drug delivery systems has not been found to be promising for effective and efficacious drug delivery. The main disadvantage of such Phospholipids based drug delivery system is the inconsistency of phospholipids which may lead to affect the stability of complexation. Chitosan and its derivatives are used to improve the bioavailability of the natural bioactive molecules. However, the drawback of the above-mentioned polymer is its fast dissolution in the stomach and its derivatives may lead to unknown chemical entities.

Many existing technologies use complexation technology to enhance the bioavailability of the natural product. However, the bioavailability of the natural product is limited due to the instability of the complexed natural product. Accordingly, there remains need a method for enhancing the bioavailability of the natural bioactive molecules by complexing it with biopolymers.

### SUMMARY OF THE INVENTION

In view of a foregoing, an embodiment herein provides a method of preparing a composition for increasing bioavailability of a natural bioactive molecule upon administration of the composition. The method includes (i) obtaining a natural bioactive molecule at a concentration of 70 to 90% by weight, (ii) blending the natural bioactive molecule at a room temperature for a time period of 1 to 3 hour, (iii) wetting blended natural bioactive molecule with a wetting agent at the room temperature for a time period of 1 to 3 hour, (iv) mixing wetted natural bioactive molecule with a biopolymer at a concentration of 10 to 30% by weight to associate the natural bioactive molecule with the biopolymer through hydrogen bonding or molecular interaction for preparing the composition as a complex of natural bioactive molecule and biopolymer, (v) drying the complex of natural bioactive molecule and biopolymer at a temperature of 110 to 130 degree Celsius (° C.), and (vi) granulating dried complex of natural bioactive molecule and biopolymer to form a pharmaceutically acceptable dosage form, wherein the biopolymer associated with the natural bioactive molecule increases bioavailability of the natural bioactive molecule upon administration.

In an embodiment, the wetting agent includes water and Isopropyl alcohol in 85:15 ratio.

In another embodiment, the biopolymer includes cellulose derivative.

In yet another embodiment, the cellulose derivative is a microcrystalline cellulose powder.

In yet another embodiment, the cellulose derivative is in a size range of 10 to 50 micrometers (μm).

In yet another embodiment, the natural bioactive molecule includes at least one form of nutraceuticals, cosmetics, food supplements, or detoxifiers.

In yet another embodiment, the natural bioactive molecule is selected from the group comprising of Shatavari extract, Safed musli, Pomegranate extract, Sunflower lecithin, Selenium, or mixture thereof.

In yet another embodiment, the natural bioactive molecule is in natural raw form.

In yet another embodiment, the pharmaceutically acceptable dosage form comprises at least one of a solid dosage form, a liquid dosage form or a semisolid dosage form.

In yet another embodiment, the pharmaceutically acceptable dosage form is at least one of an oral dosage form, or an injectable dosage form.

In yet another embodiment, the complex of natural bioactive molecule and biopolymer includes 80% by wt. of the natural molecule and 20% by wt. of the cellulose derivatives.

In one aspect, a method of preparing a composition for increasing bioavailability of a natural bioactive molecule upon administration of the composition is provided. The method includes (i) obtaining a natural bioactive molecule including at least one of Shatavari extract, Safed musli, Pomegranate extract, Sunflower lecithin, or Selenium at a concentration of 70 to 90% by weight, (ii) blending the natural bioactive molecule at a room temperature for a time period of 1 to 3 hour, (iii) wetting blended natural bioactive molecule with a wetting agent at the room temperature for a time period of 1 to 3 hour, (iv) mixing wetted natural bioactive molecule with microcrystalline cellulose powder at a concentration of 10 to 30% by weight to associate the natural bioactive molecule with the microcrystalline cellulose powder through hydrogen bonding or molecular interaction for preparing the composition as a complex of natural bioactive molecule and microcrystalline cellulose powder, (v) drying the complex of natural bioactive molecule and microcrystalline cellulose powder at a temperature of 110 to 130 degree Celsius (° C.), and (vi) granulating dried complex of natural bioactive molecule and microcrystalline cellulose powder to form a pharmaceutically acceptable dosage form, wherein the microcrystalline cellulose powder associated with the natural bioactive molecule increases bioavailability of the natural bioactive molecule upon administration.

In another aspect, a composition for increasing bioavailability of a natural bioactive molecule upon administration is provided. The composition includes 80% by wt. of the natural bioactive molecule, and 20% by wt. of a microcrystalline cellulose powder in a size of 10 to 50 μm, wherein the natural bioactive molecule is integrated with the microcrystalline cellulose powder through hydrogen bonding or molecular interaction and complex of natural bioactive molecule and microcrystalline cellulose powder is absorbed into bodily fluids as a single entity and dissociated after absorption that consequently improves the bioavailability of the natural bioactive molecule. The natural bioactive molecule includes at least one of Shatavari extract, Safed musli, Pomegranate extract, Sunflower lecithin, or Selenium.

In an embodiment, the wetting agent includes water and Isopropyl alcohol in 85:15 ratio.

In another embodiment, the method of preparing the composition for increasing bioavailability of the natural bioactive molecule upon administration of the composition is a simple process and devoid of any complex technology.

In yet another embodiment, the natural bioactive molecules are used in raw natural form to enhance the biological activities of the natural bioactive molecule.

In yet another embodiment, the biopolymer tightly holds the natural bioactive molecule until enter into the bodily fluids and disassociated from the natural bioactive molecule after entering into the bodily fluids.

### BRIEF DESCRIPTION OF THE DRAWINGS

The embodiments herein will be better understood from the following detailed description with reference to the drawings, in which:

FIG. 1 is a flow diagram illustrating a method of complexing a biopolymer with a natural bioactive molecule using a biopolymer complexation technology, according to an embodiment herein;

FIG. 2 illustrates a graphical representation of complex of natural bioactive molecule with cellulose derivatives, according to an exemplary embodiment herein;

FIG. 3 is a graphical illustration that depicts effect biopolymer complexation (BPC) enabled nutraceutical product on serum estradiol level, according to an exemplary embodiment herein;

FIG. 4 is a graphical illustration that depicts effect biopolymer complexation (BPC) enabled nutraceutical product on serum glutamate oxaloacetate transaminase (SGOT) and serum glutamate pyruvate transaminase (SGPT) levels, according to an exemplary embodiment herein;

FIG. 5 is a graphical illustration that depicts effect biopolymer complexation (BPC) enabled nutraceutical product on female sexual function inventory (FSFI) Scale or Index, according to an exemplary embodiment herein; and

FIG. 6 is a graphical illustration that depicts female sexual function inventory (FSFI) Scale or Index on placebo group, according to an exemplary embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

As mentioned, there remains a need for a method for enhancing bioavailability of the natural bioactive molecules. The embodiments herein achieve this by proposing a method of preparing a composition as a complex of natural bioactive molecule and cellulose derivatives for increasing the bioavailability of the natural bioactive molecule.

The chemistry of cellulose shows that this macromolecule not only forms the foundation of the plants and trees but also tightly holds the active ingredients or key functional molecules within the macromolecule regions. Cellulose is the most abundant biopolymer on earth. Cellulose has an inexhaustible polymeric raw material with fascinating structure and properties. Formed by the repeated connection of D-glucose building blocks, the highly functionalized, the linear stiff-chain homopolymer is characterized by its hydrophilicity, chirality, biodegradability, broad chemical modifying capacity, and its formation of versatile semi-crystalline fiber morphologies. Cellulose is the building blocks of the plant kingdom contributing more than 60% in nature and it is low in cost. This interesting nature of the cellulose is observed and deployed identical characteristics to make Biopolymer Complexation Product (BPC Products).

The active ingredients/bioactive molecules and the phytochemicals in the herbs and the medicinal plants are well complexed using cellulose macromolecules. Generally, the biological activities of the bioactive molecules are enhanced when used in raw form as evidenced from the traditional Ayurveda medicine and this concept of integrating natural bioactive molecules with cellulose has been possible.

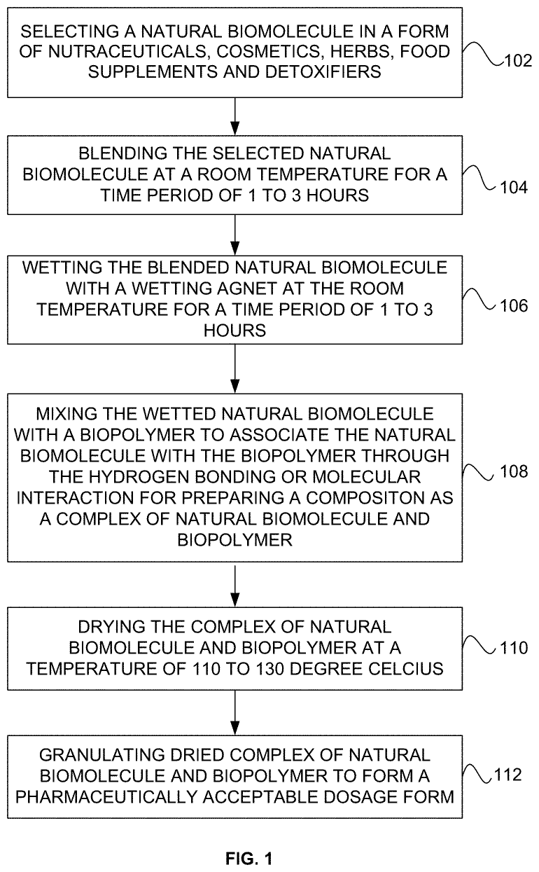

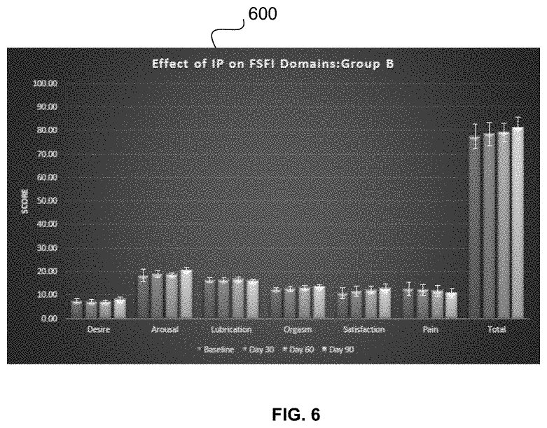

Referring now to the drawings and more particularly to FIG. 1 and FIG. 6, where similar reference characters denote corresponding features consistently throughout the figures, there are shown preferred embodiments.

FIG. 1 is a flow diagram illustrating a method of complexing a biopolymer with a natural bioactive molecule using a biopolymer complexation technology, according to an embodiment herein. At step 102, the natural bioactive molecule in the form of nutraceuticals, cosmetics, herbs & food supplements, and detoxifiers is selected. In some embodiments, the natural bioactive molecule includes a concentration of 70 to 90% by weight. In some embodiments, the natural bioactive molecule includes a concentration of 80% by weight. At step 104, the selected natural bioactive molecule is blended using a blender to obtain a blended natural bioactive molecule. In an embodiment, the blender is selected from the group including a V-blender or a double cone blender. In some embodiments, the selected natural bioactive molecule is blended in a dry chamber using the blender at a room temperature for a time period of 1 to 3 hours. In some embodiments, the selected natural bioactive molecule is blended in a dry chamber using the blender at the room temperature for the time period of 2 hours.

At step 106, the blended natural bioactive molecule is treated with wetting agents using a wetting process. In an embodiment, the wetting agents are selected from the group including anionic or cationic surfactants. In an embodiment, the blended natural bioactive molecule is treated with the wetting agents to form a slurry, wet granulation or paste mixture of the blended natural bioactive molecule with the wetting solution. In an embodiment, the wetting process is performed by adding small increments of the wetting solution to the blended natural bioactive molecule. In an embodiment, when the natural bioactive molecule is an amorphous or crystalline, the wetting agent crystallizes amorphous regions of the natural bioactive molecule into ultra-micro size crystals and splits the clusters of such crystals for dispersing spontaneously when exposed to water. In an embodiment, the wetting agent comprises an aqueous solution which distributes the wetting agent to surfaces of the natural bioactive molecule, when the natural bioactive molecule is not amorphous or crystalline. In some embodiments, the wetting agents include water and Isopropyl alcohol in 85:15 ratio. In some embodiments, the blended natural bioactive molecule is treated with wetting agents at the room temperature for a time period of 1 to 3 hour. In some embodiments, the blended natural bioactive molecule is treated with water and Isopropyl alcohol in 85:15 ratio at the room temperature for the time period of 2 hours.

At step 108, cellulose material in the form of cellulose derivatives are added to the wetted natural bioactive molecule. In some embodiments, the cellulose derivatives include a concentration of 10 to 30% by weight. In some embodiments, the cellulose derivatives include a concentration of 20% by weight. The cellulose derivatives include at least one of, but not limited to, cellulose ester, cellulose ethers, hydroxyl propyl methylcellulose, hydroxyl ethyl cellulose, hydroxyl propyl cellulose, hydroxy methylcellulose, methyl cellulose, sodium carboxymethyl cellulose, carboxymethylcellulose, cellulose acetate, microcrystalline cellulose triacetate, cellulose nanowhiskers, or quaternized cellulose. In some embodiments, the cellulose derivative is a microcrystalline cellulose powder. In some embodiments, the cellulose derivative is in a size range of 10 to 50 micrometers (μm). In some embodiments, the cellulose derivative is in a size range of 20 to 30 μm. In some embodiments, the cellulose derivative at the concertation of 10 to 30% by weight is added to the wetted natural bioactive molecule to associate the natural bioactive molecule into the cellulose derivatives through hydrogen bonding or molecular interaction for preparing the composition as a complex of natural bioactive molecule and cellulose derivatives.

In some embodiment, the composition includes 80% by weight of the natural bioactive molecule and 20% by weight of a microcrystalline cellulose powder, wherein the natural bioactive molecule is integrated with the microcrystalline cellulose powder through hydrogen bonding or molecular interaction and complex of natural bioactive molecule and microcrystalline cellulose powder is absorbed into bodily fluids as a single entity and dissociated after absorption that consequently improves the bioavailability of the natural bioactive molecule. At step 110, the liquid mixture of the natural bioactive molecule and the cellulose material is atomized into a hot air stream in a spray dryer and the hot air stream rapidly dry the liquid mixture of the natural bioactive molecule and the cellulose material and forms a powder form of natural bioactive molecule-cellulose mixture. The spray-dryer comprises a drying chamber, an atomizing means for atomizing a solvent-containing the liquid mixture of natural bioactive molecule and the cellulose material feed into the drying chamber, a source of heated drying gas/hot air stream directed into the drying chamber and a dried product collection means for separating the dried product from the cooled drying gas and vaporized solvent stream, following its exit from the drying chamber. The spray dryers may be a single stage dryer, a two-stage dryer, a mixed flow dryer, a vertical dryer, a horizontal dryer, a closed cycle dryer, or a semi-closed dryer. In some embodiments, the liquid mixture of the natural bioactive molecule and the cellulose material is dried at a temperature of 110 to 130 degree Celsius (° C.). In some embodiments, the liquid mixture of the natural bioactive molecule and the cellulose material is dried at a temperature of 130° C.

In one embodiment, the natural bioactive molecule in the form of but not limited to, nutraceuticals, cosmetics, herbs, food supplements, and antioxidants, or combinations thereof.

At step 112, dried complex of the natural bioactive molecule and cellulose derivate is granulated to form a pharmaceutically acceptable dosage form. The cellulose derivate associated with the natural bioactive molecule increases bioavailability of the natural bioactive molecule upon administration.

In some embodiments, the pharmaceutically acceptable dosage form comprises at least one of a solid dosage form, a liquid dosage form or a semisolid dosage form.

In some embodiments, the pharmaceutically acceptable dosage form is at least one of an oral dosage form, or an injectable dosage form.

In some embodiments, the complex of natural bioactive molecule and cellulose derivate is stable at room temperature.

In some embodiments, the complex of natural bioactive molecule and cellulose derivate is stored under room temperature.

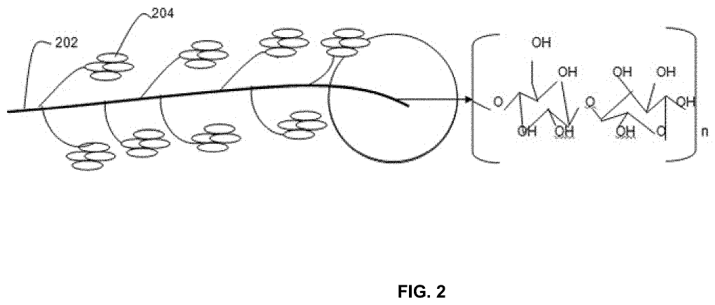

FIG. 2 illustrates a graphical representation of complex of natural bioactive molecule with cellulose derivatives, according to an exemplary embodiment herein. The cellulose derivative 202 is surface modified with the natural bioactive molecules 204 by forming a hydrogen bonding or an ionic interaction between functional groups of the cellulose derivative 202 and the natural bioactive molecules 204 between the cellulose derivative 202 and the natural bioactive molecules 204 to form a complex of the cellulose derivative 202 and the natural bioactive molecules 204. The complex of the cellulose derivative 202 and the natural bioactive molecules 204 is easily absorbed to the blood as a single entity and the complex is dissociated once the complex is absorbed.

In an exemplary embodiment, a method of preparing a nutraceutical product using a biopolymer complexation technology for treating sexual dysfunction in women is provided. The method includes (i) obtaining nutraceutical compounds selected from a group including of Shatavari extract, Safed musli, Pomegranate extract, Sunflower lecithin, Selenium, or combinations thereof at a concentration range from 70 to 90% by weight, (ii) blending the nutraceutical compounds at a room temperature for a time period of 1 to 3 hour, (iii) wetting blended nutraceutical compounds with water and Isopropyl alcohol (85:15 ratio) at the room temperature for a time period of 1 to 3 hour, (iv) mixing wetted nutraceutical compounds with microcrystalline cellulose powder at a concentration of 10 to 30% by weight to associate the nutraceutical compounds with the microcrystalline cellulose powder through hydrogen bonding or molecular interaction for preparing the nutraceutical product as a complex of nutraceutical compounds and microcrystalline cellulose powder, (v) drying the complex of nutraceutical compounds and microcrystalline cellulose powder at a temperature of 110 to 130 degree Celsius (° C.), and (vi) granulating dried complex of nutraceutical compounds and microcrystalline cellulose powder to form a pharmaceutically acceptable dosage form. The microcrystalline cellulose powder associated with the nutraceutical compounds increases bioavailability of the nutraceutical compounds upon administration. The microcrystalline cellulose powder is purchased from Lab Chem Solutions Bangalore, Karnataka, India. The source of microcrystalline cellulose powder is cellulose from Cotton (Gossypium malvaceae). The nutraceutical compounds Shatavari extract, Safed musli, Pomegranate extract are purchased from Mehta Dyechem, Bangalore, India. The Sunflower lecithin is purchased from Shankar Nutrition, Madhya Pradesh, India

For example, the nutraceutical product includes

TABLE 1

| S. No | Active Ingredients | Quantity (mg) |
|---|---|---|
| 1 | Shatavari extract | 100 to 200 |
| 2 | Musli | 75 to 175 |
| 3 | Pomegranate extract | 25 to 125 |
| 4 | Sunflower Lecithin | 10 to 65 |
| 5 | Selenium | 1 to 10 |
| 6 | Cellulose | 50 to 150 |

In an exemplary embodiment, the nutraceutical product as synthesized through the bio-complexation technology (BPC) has been investigated clinically for its efficacy on treating sexual dysfunction in women. Clinical assessments of effect of nutraceutical product supplement on primary and secondary efficacy variables in comparison with placebo are performed at scheduled time points. The scheduled time points include day 1 (baseline), day 30, day 60 and day 90. Efficacy evaluations in this study are done by objective as well as subjective assessments. In this study 24 subjects are considered wherein 12 subjects consumed nutraceutical product and another 12 subjects consumed placebo. Two efficacy data points and five safety data points are assessed throughout the study. The efficacy data points include serum estradiol and female sexual function index (FSFI). The safety data points include serum glutamate Pyruvate Transaminase (SGPT), serum glutamate oxaloacetate transaminase (SGOT), hematological parameters, heart rate (HR), blood pressure (BP) and body temperature. The following tables provide values of efficacy data points and safety data points assessed throughout the study.

Table 2 includes data points for the parameters serum estradiol, FSFI, SGOT, and SGPT that are assessed for nutraceutical product consumed group (Group A).

Nutraceutical product consumed group
(N = 12 subjects) - Group A

| | Baseline | | | |
|---|---|---|---|---|
| | Day 1 | Day 30 | Day 60 | Day 90 |
| Parameters | Mean ± SD | Mean ± SD | Mean ± SD | Mean ± SD |
| serum | 126.71 ± | 94.38 ± | 105.14 ± | 112.86 ± |
| estradiol | 74.08 | 90.10 | 62.11 | 86.03 |
| (pg/mL) | | | | |
| P (within | — | 0.34 | 0.52 | 0.22 |
| group) | | | | |
| FSFI Total | 76.00 ± | 71.67 ± | 65.25 ± | 62.25 ± |
| Score | 4.28 | 5.59 | 4.73 | 2.95 |
| P (within | — | 0.001 | <0.001 | <0.001 |
| group) | | | | |
| SGOT | 21.48 ± | — | — | 16.61 ± |
| (U/L) | 9.04 | | | 2.07 |
| P (within | — | — | — | 0.38 |
| group) | | | | |
| SGPT | 15.11 ± | | | 14.15 ± |
| (U/L) | 11.81 | | | 5.14 |
| P (within | — | — | — | 0.13 |
| group) | | | | |

Table 3 includes data points for the parameters serum estradiol, FSFI, SGOT, and SGPT that are assessed for placebo group (Group B)

Placebo group (N = 12 subjects) - Group B

| | Baseline | | | |
|---|---|---|---|---|
| | Day 1 | Day 30 | Day 60 | Day 90 |
| Parameters | Mean ± SD | Mean ± SD | Mean ± SD | Mean ± SD |
| Serum | 138.09 ± | 83.76 ± | 91.18 ± | 101.38 ± |
| Estradiol | 119.12 | 73.18 | 77.37 | 121.93 |
| (pg/mL) | | | | |
| P (within | — | 0.23 | 0.35 | 0.56 |
| group) | | | | |
| FSFI Total | 77.50 ± | 78.58 ± | 79.25 ± | 81.17 ± |
| Score | 5.44 | 4.96 | 3.94 | 4.56 |
| P (within | — | 0.41 | 0.40 | 0.12 |
| group) | | | | |
| SGOT | 23.75 ± | — | — | 18.40 ± |
| (U/L) | 11.07 | | | 3.91 |
| P (within | — | — | — | 0.17 |
| group) | | | | |
| SGPT | 22.28 ± | — | — | 14.14 ± |
| (U/L) | 16.57 | | | 5.22 |
| P (within | — | — | — | 0.11 |
| group) | | | | |

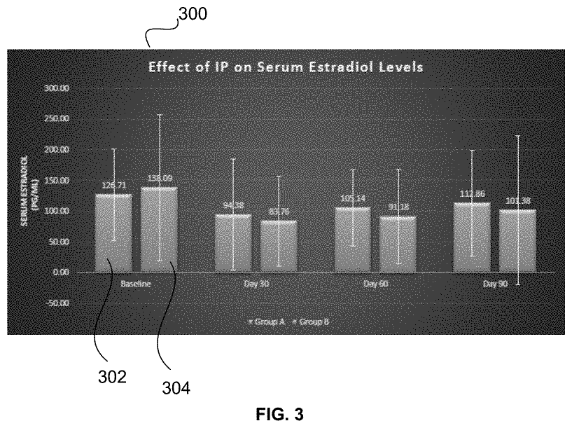

FIG. 3 is a graphical illustration 300 that depicts effect biopolymer complexation (BPC) enabled nutraceutical product on serum estradiol level, according to an exemplary embodiment herein. The graphical illustration 300 has serum estradiol level in Y-axis plotted against time point in X-axis. The graphical illustration 300 compares serum estradiol level between BPC enabled nutraceutical product consumed group (Group A) 302 and placebo group (Group B) 304 to determine the effect BPC enabled nutraceutical product on the serum estradiol level. The graphical illustration 300 depicts that serum estradiol level is increased over 90 days period in BPC enabled nutraceutical product consumed group (Group A) 302.

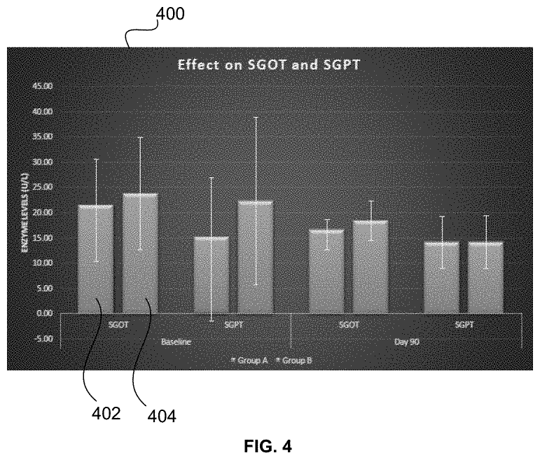

FIG. 4 is a graphical illustration 400 that depicts effect biopolymer complexation (BPC) enabled nutraceutical product on serum glutamate oxaloacetate transaminase (SGOT) and serum glutamate pyruvate transaminase (SGPT) levels, according to an exemplary embodiment herein. The graphical illustration 400 has SGOT and SGPT levels in Y-axis plotted against time point in X-axis. The graphical illustration 400 compares SGOT and SGPT levels between BPC enabled nutraceutical product consumed group (Group A) 402 and placebo group (Group B) 404 to determine effect BPC enabled nutraceutical product on SGOT and SGPT levels. The graphical illustration 400 depicts that SGOT and SGPT levels did not show any significant changes over 90 days period in BPC enabled nutraceutical product consumed group (Group A) 402 as compared to placebo group (Group B) 404.

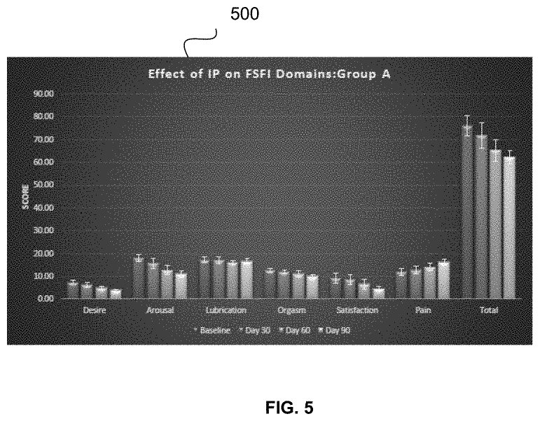

FIG. 5 is a graphical illustration 500 that depicts effect biopolymer complexation (BPC) enabled nutraceutical product on female sexual function inventory (FSFI) Scale or Index, according to an exemplary embodiment herein. The graphical illustration 500 has FSFI score in Y-axis plotted against time point in X-axis.

FIG. 6 is a graphical illustration 600 that depicts female sexual function inventory (FSFI) Scale or Index on placebo group, according to an exemplary embodiment herein. The graphical illustration 600 has FSFI score in Y-axis plotted against time point in X-axis.

It is clearly evident from the Table 2, and FIG. 3 that the investigational nutraceutical product is effective in bringing back the serum estradiol levels close to original value by the end of 60 days and maintained serum estradiol to elevated level till the end of 90 days. In placebo group as given in Table 3 and in FIG. 3, there is decrease in serum estradiol levels till the end of the study when compared with baseline values. The clinical results affirmed that the bio-complexation (BPC) enabled nutraceutical product is capable of maintaining the serum estradiol levels to original baseline value. Similarly, with reference to FIG. 5 and FIG. 6, the nutraceutical product improves the sexual function in all the individual domains as well as in the total value. In the placebo group, all of the FSFI domains could observe deterioration in the sexual function. With reference to FIG. 4, the safety variables SGOT and SGPT did not show any statistically or clinically significant change over a period of 90 days, suggesting safe for clinical use without causing any liver damage.

Table 4 includes data points for haematological parameters that are assessed for BPC enabled nutraceutical product consumed group (Group A) and placebo group (Group B). All of the hematological parameters lied within the normal clinical range for both group A and group B assessed at the beginning and end of the study. Intergroup p value is significant only on case of number of neutrophils due to significantly different baseline values and not due to any effect from BPC enabled nutraceutical product.

| | Group A | | | | Group B | | | | |
|---|---|---|---|---|---|---|---|---|---|
| | | | End of study | | | | End of study | | |
| | Baseline | | (Day 90) | | Baseline | | (Day 90) | | |
| Parameters | Mean | SD | Mean | SD | Mean | SD | Mean | SD | P |
| Hb (g/dL) | 12.60 | 1.09 | 12.67 | 1.24 | 12.36 | 1.09 | 12.08 | 1.06 | 0.24 |
| PCV (%) | 37.78 | 2.88 | 38.53 | 3.70 | 37.46 | 3.15 | 37.30 | 2.56 | 0.37 |
| Total | 7670.00 | 1182.41 | 7055.0 | 2269.6 | 7316.67 | 2087.2 | 14183.33 | 23826.49 | 0.34 |
| Leukocyte | | | | | | | | | |
| Count | | | | | | | | | |
| (10\hat{ }6/L) | | | | | | | | | |
| RBC Count | 4.41 | 0.40 | 4.58 | 0.40 | 4.47 | 0.37 | 4.45 | 0.34 | 0.43 |
| (mill/mm3) | | | | | | | | | |
| MCV (FL) | 86.00 | 7.34 | 86.33 | 6.55 | 84.25 | 6.06 | 85.58 | 8.31 | 0.82 |
| MCH (pg) | 28.68 | 2.77 | 28.21 | 2.53 | 27.81 | 2.62 | 26.00 | 5.82 | 0.27 |
| MCHC | 33.32 | 0.96 | 32.83 | 1.32 | 33.03 | 0.99 | 32.38 | 1.51 | 0.46 |
| (g/dL) | | | | | | | | | |
| RDW (%) | 14.56 | 1.25 | 14.55 | 1.62 | 14.93 | 1.72 | 14.46 | 1.71 | 0.90 |
| Platelet | 290700 | 94973 | 325100 | 46319 | 305667 | 79073 | 333250 | 51861 | 0.71 |
| count | | | | | | | | | |
| (thou/mm3) | | | | | | | | | |
| Neutrophils | 50.98 | 7.07 | 52.17 | 6.77 | 56.23 | 6.97 | 58.49 | 6.88 | 0.04 |
| (%) | | | | | | | | | |
| Lymphocyte | 39.23 | 6.92 | 37.93 | 6.31 | 32.44 | 4.33 | 31.53 | 5.10 | 0.02 |
| (%) | | | | | | | | | |
| Monocytes | 7.04 | 1.24 | 6.39 | 1.72 | 6.65 | 1.51 | 11.73 | 17.63 | 0.34 |
| (%) | | | | | | | | | |
| Eosinophils | 2.68 | 1.37 | 2.48 | 1.17 | 4.66 | 3.72 | 3.88 | 2.57 | 0.12 |
| (%) | | | | | | | | | |
| Basophils | 0.08 | 0.15 | 0.12 | 0.15 | 0.02 | 0.06 | 0.04 | 0.06 | 0.15 |
| (%) | | | | | | | | | |
| Serum | 0.61 | 0.11 | 0.62 | 0.09 | 0.67 | 0.09 | 0.64 | 0.08 | 0.65 |
| Creatinine | | | | | | | | | |
| AST | 21.48 | 9.04 | 16.61 | 2.07 | 23.75 | 11.07 | 18.40 | 3.91 | 0.21 |
| (SGOT) | | | | | | | | | |
| U/L | | | | | | | | | |
| ALT | 15.11 | 11.81 | 14.15 | 5.14 | 22.28 | 16.57 | 14.14 | 5.22 | 1.00 |
| (SGPT) | | | | | | | | | |
| U/L | | | | | | | | | |
| Estradiol | 126.71 | 74.08 | 112.86 | 86.03 | 138.09 | 119.12 | 101.38 | 121.93 | 0.80 |
| (E2) | | | | | | | | | |
| pg/m:L | | | | | | | | | |

Table 5 includes data points for HR and BP that are assessed for BPC enabled nutraceutical product consumed group (Group A) and placebo group (Group B). Both group A and group B exhibits the vital parameter values within the clinically normal range throughout the study period.

| | Group A | | | | Group B | | | | |
|---|---|---|---|---|---|---|---|---|---|
| Parameters | Day 1 | Day 30 | Day 60 | Day 90 | Day 1 | Day 30 | Day 60 | Day 90 | P |
| SBP (mmHg) | 118.8 ± 1.28 | 119.2 ± 1.3 | 119.8 ± 1.2 | 118.1 ± 1.9 | 119.1 ± 0.9 | 119.4 ± 1.2 | 119.3 ± 1.4 | 118.7 ± 2.1 | 0.50 |
| DBP (mmHg) | 79.8 ± 0.99 | 79.7 ± 0.7 | 79.5 ± 1.4 | 79.1 ± 1.2 | 79.8 ± 0.5 | 79.8 ± 0.9 | 80.3 ± 0.7 | 78.6 ± 1.2 | 0.36 |
| HR (Heartbeats | 77.5 ± 3.28 | 78.2 ± 2.5 | 77.3 ± 3.5 | 77.5 ± 2.6 | 76.0 ± 4.0 | 77.3 ± 3.0 | 78.6 ± 1.8 | 76.5 ± 5.1 | 0.57 |
| per minute) | | | | | | | | | |
| Temperature | 97.4 ± 0.49 | 97 ± 0.4 | 97.2 ± 0.4 | 96.7 ± 0.7 | 97.4 ± 0.5 | 97.5 ± 0.5 | 97.4 ± 0.4 | 97.0 ± 0.4 | 0.27 |
| (°F.) | | | | | | | | | |

The results of clinical investigation of the nutraceutical product clearly indicating that better results have been obtained in terms of improving sexual dysfunction in women after incorporating bio-complexation technology. The results of BPC enabled nutraceutical product indicate that the nutraceutical product consumed group demonstrated greater efficacy in all variables than the placebo group. The BPC enabled nutraceutical product has stabilizing effect on serum estradiol levels and very strong positive effect on all attributes of Female Sexual Function Inventory scales. The clinical investigation of the nutraceutical product provides initial evidence that the BPC enabled nutraceutical product is indeed useful for treatment of Hypoactive sexual desire disorder (HSDD) in women with age range from 25-55 years and has the potential for normalizing the female sexual health without any adverse effects.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A method of preparing a composition for increasing bioavailability of a natural bioactive molecule upon administration of the composition, wherein said method comprising;
obtaining a natural bioactive molecule at a concentration of 70 to 90% by weight;
blending the natural bioactive molecule at a room temperature for a time period of 1 to 3 hour;
wetting blended natural bioactive molecule with a wetting agent at the room temperature for a time period of 1 to 3 hour, wherein the wetting agent comprises water and Isopropyl alcohol in 85:15 ratio;
mixing wetted natural bioactive molecule with a biopolymer at a concentration of 10 to 30% by weight to associate the natural bioactive molecule with the biopolymer through hydrogen bonding or molecular interaction for preparing the composition as a complex of natural bioactive molecule and biopolymer, wherein the biopolymer comprises cellulose derivative;
drying the complex of natural bioactive molecule and biopolymer at a temperature of 110 to 130 degree Celsius (° C.); and
granulating dried complex of natural bioactive molecule and biopolymer to form a pharmaceutically acceptable dosage form, wherein the biopolymer associated with the natural bioactive molecule increases bioavailability of the natural bioactive molecule upon administration.

2. The method as claimed in claim 1, wherein the cellulose derivative is a microcrystalline cellulose powder.

3. The method as claimed in claim 1, wherein the cellulose derivative is in a size range of 10 to 50 micrometers (μm).

4. The method as claimed in claim 1, wherein the natural bioactive molecule comprises at least one form of nutraceuticals, cosmetics, food supplements, or detoxifiers.

5. The method as claimed in claim 1, wherein the natural bioactive molecule is selected from the group comprising of Shatavari extract, Safed musli, Pomegranate extract, Sunflower lecithin, Selenium, or mixture thereof.

6. The method as claimed in claim 1, wherein the natural bioactive molecule is in natural raw form.

7. The method as claimed in claim 1, wherein the pharmaceutically acceptable dosage form comprises at least one of a solid dosage form, a liquid dosage form or a semisolid dosage form.

8. The method as claimed in claim 1, wherein the pharmaceutically acceptable dosage form is at least one of an oral dosage form, or an injectable dosage form.

9. The method as claimed in claim 8, wherein the oral dosage form is a capsule form.

10. The method as claimed in claim 1, wherein the complex of natural bioactive molecule and biopolymer comprises 80% by weight of the natural bioactive molecule and 20% by weight of the cellulose derivatives.

11. The method as claimed in claim 1, wherein the method comprises storing pharmaceutically acceptable dosage form of complex of natural bioactive molecule and biopolymer under room temperature.

12. A method of preparing a composition for increasing bioavailability of a natural bioactive molecule upon administration of the composition, wherein said method comprising;
obtaining a natural bioactive molecule comprising at least one of Shatavari extract, Safed musli, Pomegranate extract, Sunflower lecithin, or Selenium at a concentration of 70 to 90% by weight;
blending the natural bioactive molecule at a room temperature for a time period of 1 to 3 hour;
wetting blended natural bioactive molecule with a wetting agent at the room temperature for a time period of 1 to 3 hour, wherein the wetting agent comprises water and Isopropyl alcohol in 85:15 ratio;
mixing wetted natural bioactive molecule with microcrystalline cellulose powder at a concentration of 10 to 30% by weight to associate the natural bioactive molecule with the microcrystalline cellulose powder through hydrogen bonding or molecular interaction for preparing the composition as a complex of natural bioactive molecule and microcrystalline cellulose powder;
drying the complex of natural bioactive molecule and microcrystalline cellulose powder at a temperature of 110 to 130 degree Celsius (° C.); and
granulating dried complex of natural bioactive molecule and microcrystalline cellulose powder to form a pharmaceutically acceptable dosage form, wherein the microcrystalline cellulose powder associated with the natural bioactive molecule increases bioavailability of the natural bioactive molecule upon administration.

13. A composition for increasing bioavailability of a natural bioactive molecule upon administration, wherein said composition comprising;
(i) 80% by wt. of the natural bioactive molecule comprising at least one of Shatavari extract, Safed musli, Pomegranate extract, Sunflower lecithin, or Selenium; and
(ii) 20% by wt. of a microcrystalline cellulose powder in a size of 10 to 50 μm, wherein the natural bioactive molecule is integrated with the microcrystalline cellulose powder through hydrogen bonding or molecular interaction and complex of natural bioactive molecule and microcrystalline cellulose powder is absorbed into bodily fluids as a single entity and dissociated after absorption that consequently improves the bioavailability of the natural bioactive molecule.

14. The composition as claimed in claim 13, wherein the natural bioactive molecule is in natural raw form.

15. The composition as claimed in claim 13, wherein the composition is in at least one of an oral dosage form, or an injectable dosage form.

16. The composition as claimed in claim 15, wherein the oral dosage form is a capsule form.

17. The composition as claimed in claim 13, wherein the composition is stored under room temperature.
